(19) United States
(12) Patent Application Publication
(10) Pub. No.: US 2025/0082707 A1
(43) Pub. Date: Mar. 13, 2025

(54) COMPOSITION FOR PREVENTION AND TREATMENT OF OBESITY AND SARCOPENIA, COMPRISING RHODODENDRON MUCRONULATUM EXTRACT AS ACTIVE INGREDIENT

(71) Applicant: DR. OREGONIN INC., Chuncheon-si (KR)

(72) Inventor: Sun Eun CHOI, Chuncheon-si (KR)

(21) Appl. No.: 18/728,029

(22) PCT Filed: May 10, 2022
(86) PCT No.: PCT/KR2022/006679

§ 371 Date: Jul. 10, 2024

(30) Foreign Application Priority Data
Jan. 11, 2022 (KR) 10-2022-0004024
Mar. 28, 2022 (KR) 10-2022-0037908

(51) Int. Cl. A61K 36/45; A61K 31/353; A61K 31/7048

(52) U.S. Cl. CPC A61K 36/45; A61K 31/353; A61K 31/7048; A61K 2236/37

(57) ABSTRACT

Provided is a pharmaceutical composition for preventing and treating obesity and muscle loss, containing Rhododendron mucronulatum extract as an active ingredient.

### TECHNICAL FIELD

The present invention relates to a composition for preventing and treating obesity and muscle loss, which includes a Rhododendron mucronulatum extract as an active ingredient.

### BACKGROUND ART

Lipid metabolism is necessary for the storage and distribution of our body energy, control of glucose metabolism, and maintain energy homeostasis, and abnormalities in lipid metabolism can cause symptoms such as obesity, diabetes, and hyperlipidemia. This lipid metabolism mainly occurs in the liver and adipose tissue, and in adipose tissue, it is controlled by the adipocytes constituting the tissue. Adipocytes are one of the important organs in body metabolism, not just energy storage organs, but also endocrine organs that secrete various hormones, and are organs that play an active role in the metabolic process.

Adipocytes induce obesity due to an increase in the amount of triglycerides or the number of adipocytes in adipocytes. Therefore, in preventing and treating obesity, it is necessary to find a way to reduce fat accumulation and the number of adipocytes. In addition, since preadipocytes are differentiated from adipocytes, a mechanism study for adipogenesis is also very important in understanding the role of adipose tissue. Recently, molecular biological research on the differentiation of adipocytes and regulatory organs constituting adipose tissue has been extensively conducted. However, research on uncovering clear efficacy at the level of single compounds is insufficient.

Meanwhile, muscles may be divided into skeletal muscle, smooth muscle, and cardiac muscle in terms of structure or function. Among them, skeletal muscle is about 600 voluntary muscles that are directly under the skin of the hands, feet, breasts, abdomen, etc., and are attached to the bones through the bones or tendons of the whole body. It is suitable for moving or supporting bone through contraction. The contraction is caused and controlled by a neural signal. It accounts for 40-50% of the body weight and functions to maintain body temperature and generate energy. The micromyofibrils of actin and myosin are regularly arranged, so that the horizontal patterns can be observed on a microscope (Lieber R. L., 2002; Edwards R. H., 1981).

Skeletal muscle fibers are divided into three categories: Type I, Type IIa, and Type IIb by biochemical classification according to the content of mitochondria. A postural muscle that is made of red slow muscle fiber and maintains the posture by maintaining a weak force for a long time is called Type I. It is a muscle suitable for exercise such as aerobic long-distance running due to its high mitochondrial content. Among the fast muscle fibers, the one having the feature of the slow muscle fiber is called Type IIa. When making movements, muscles made of white fast muscle fibers are used, which are called active muscles and are classified as Type IIb. It is a muscle suitable for exercise such as anaerobic short-distance running due to its low mitochondrial content. These skeletal muscle fibers are distributed in different proportions for each part of the body (Tortora et al, 2008).

The anti-anabolic and catabolic action of unbalanced muscle fibers leads to muscle atrophy. Here, the muscular atrophy refers to the size and mass loss of muscle cells and muscle tissue when the muscle is not used due to aging, a disease state (excessive exposure to stress hormones, cancer, sepsis, starvation, etc.), and a decrease in activities such as pathological life. When muscle atrophy occurs, muscle strength for physical activity is weakened, and a vicious cycle of musculoskeletal degeneration begins. A decrease in walking speed and a decrease in grip strength are main symptoms and indicators of a decrease in muscle mass, and may lead to falls, fractures, joint damage, metabolic disorders, and cardiovascular diseases.

The glucocorticoids of our body causes molecular biological changes in muscle fibers and is directly or indirectly involved in anti-anabolism and catabolism. The Glucocorticoids-based compound, dexamethasone, serves to inhibit PI3K/Akt/mTOR pathway as an anti-anabolic action, which inhibits the activities of 4E-BP1 and S6K1, which are downstream effectors, thereby preventing the operation of elF4G (Eukaryotic translation initiation factor 4G) and elF4E (Eukaryotic translation initiation factor 4E). This is to inhibit the mRNA translation process for protein synthesis, which is shown as muscle fiber atrophy according to the synthesis inhibition and protein degradation of muscle fiber (Shackman et al., 2013).

In the present invention, Dexamethasone may cause synthesis inhibition and proteolysis of muscles to induce muscle atrophy. This is related to the expression of the genes atrogene (Atrogin-1, MuRF-1) that induce the muscular atrophy according to the mechanism leading to “PI3K/Akt→FOXO activation and GSK3 inactivation”, and these genes induce proteolysis represented by ubiquitin-proteasome system.

Therefore, it is necessary to develop a material for inhibiting obesity and muscle loss, which has the effect of simultaneously decomposing sarcopenia and fat, which are diseases in which skeletal muscle is reduced.

### DISCLOSURE

### Technical Problem

Therefore, the problem to be solved by the present invention is to provide an extract having an excellent anti-obesity effect and an excellent effect of preventing muscle cell loss, and a single compound obtained therefrom.

### Technical Solution

In one aspect, the present invention provides a pharmaceutical composition for preventing and treating obesity and muscle loss, which comprises Rhododendron mucronulatum extract as an active ingredient.

In another aspect, the Rhododendron mucronulatum extract comprises a taxifolin glycoside or a taxifolin aglycone.

In another aspect, the Rhododendron mucronulatum extract is obtained by supercritical extraction of Rhododendron root.

In another aspect, the taxifolin glycoside contains a compound of formula (1).

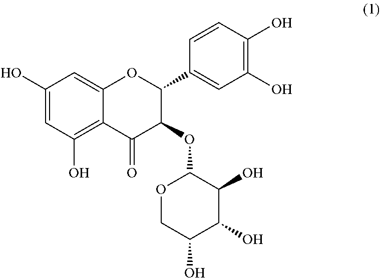

In another aspect, the taxifolin glycoside contains a compound of formula (2).

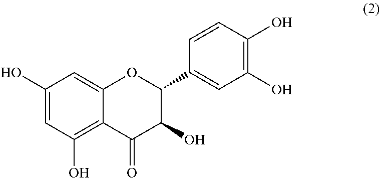 [SMILES: O=C1c2c(O)cc(O)cc2O[C@H](c2ccc(O)c(O)c2)[C@H]1O]

In another aspect, there is provided a pharmaceutical composition for preventing and treating obesity and muscle loss, which comprises at least one selected from the group consisting of compounds represented by Formulae (1) and (2) below.

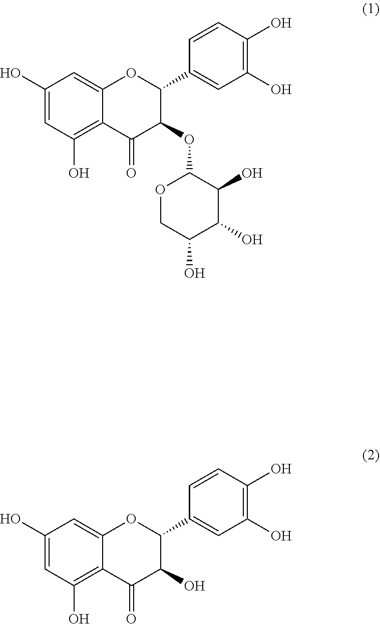

In another aspect, the compound is extracted from Rhododendron mucronulatum roots.

The present invention also provides a food composition for preventing obesity and muscle loss, which includes a Rhododendron mucronulatum extract as an active ingredient.

In another aspect, the Rhododendron mucronulatum extract comprises taxifolin glycoside or taxifolin aglycone, and the Rhododendron mucronulatum extract is obtained by supercritical extraction of Rhododendron mucronulatum roots.

In another aspect, the taxifolin glycoside contains a compound of formula (1).

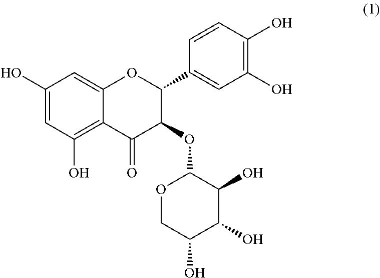

In another aspect, the taxifolin glycoside contains a compound of formula (2).

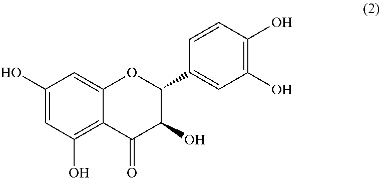 [SMILES: O=C1c2c(O)cc(O)cc2O[C@H](c2ccc(O)c(O)c2)[C@H]1O]

The present invention provides a food composition for preventing obesity and muscle-loss, which comprises one or more compounds selected from a group consisting of the compounds of formula (1) and formula (2) below.

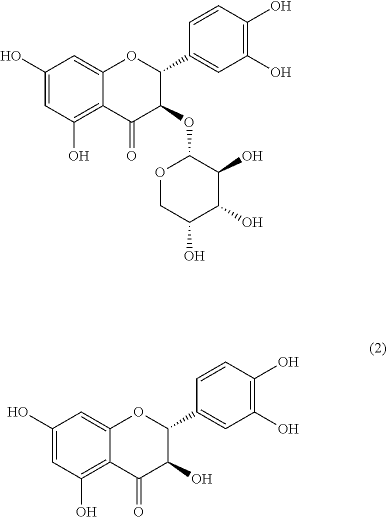 [SMILES: O=C1c2c(O)cc(O)cc2O[C@H](c2ccc(O)c(O)c2)[C@H]1O[C@H]1OC[C@@H](O)[C@@H](O)[C@@H]1O ; O=C1c2c(O)cc(O)cc2O[C@H](c2ccc(O)c(O)c2)[C@H]1O]

In another aspect, the compound is extracted from the Rhododendron mucronulatum roots.

The food composition for preventing obesity and muscle loss is also a feed additive for animals.

### Advantageous Effects

The anti-obesity composition according to the present invention is based on taxifolin glycosides and taxifolin aglycone contained in a Rhododendron mucronulatum extract, and has inhibitory activity against adipocyte differentiation in a concentration-dependent manner. In particular, at the concentration of 20 ug/ml, the highest concentration in the experiment, the effect of inhibiting adipogenesis by 40% or more was confirmed. Furthermore, the composition also has an effect of inhibiting muscle loss against hydrogen peroxide or dexamethasone.

### DESCRIPTION OF DRAWINGS

FIG. 1 is a view for explaining an extraction process according to an embodiment of the present invention.

FIG. 2 is a TLC analysis result on a high content extract (RMRF) derived from Rhododendron mucronulatum.

FIG. 3 is an analysis result of the total phenol content of a high-content extract (RMRF) derived from Rhododendron mucronulatum.

FIG. 4 shows the results of RMRF analysis of taxifolin aglycone with respect to RMRF and an alcohol extract (RM).

FIG. 5 shows the results of RMRF analysis of taxifolin glycosides of RMRF and an alcohol extract (RM).

FIG. 6 is a photograph of cells observed when the “Taxifolin-3-O-arabinopyranoside” compound identified as a marker material and an effective material of Rhododendron sp. plants, is treated.

FIG. 7 is a photograph of a cell observed when a compound in the form of non-saccharide produced through enzymatic hydrolysis, that is, a compound “Taxifolin-aglycone” is treated from a glycoside compound well known as a marker material and an effective material derived from Rhododendron sp. plants, that is, “Taxifolin-3-O-arabinopyranoside”.

FIGS. 8 and 9 are the results of analyzing the cell viability of taxifolin glycosides (RM) and taxifolin aglycone (RMRF), respectively.

FIG. 10 and FIG. 11 show the results of analyzing cell viability when taxifolin glycoside (RM) and taxifolin aglycone (RMRF) for H2O2 (hydrogen peroxide) were used, respectively.

FIG. 12 and FIG. 13 show the results of analyzing the cell viability when taxifolin glycoside (RM) and taxifolin aglycone (RMRF) were used for dexamethasone, respectively.

### BEST MODE

Hereinafter, specific embodiments of the present invention will be described with reference to the drawings. However, this is only an example, and the present invention is not limited thereto.

In describing the present invention, when it is determined that a detailed description of a known technology related to the present invention may unnecessarily obscure the gist of the present invention, the detailed description thereof will be omitted. Further, terms to be described below are terms defined in consideration of functions in the present disclosure, and may be changed according to intentions or customs of a user or an operator. Therefore, the definition should be made based on the contents throughout this specification.

The technical spirit of the present invention is determined by Claims, and the following embodiments are merely a means for efficiently explaining the technical spirit of the present invention to those skilled in the art to which the present invention pertains. The present invention provides a composition for preventing or treating hair loss, which includes a plant extract of Rhododendron mucronulatum as an active ingredient.

The present invention provides a lipid degradation inhibitor of taxifolin glycoside and taxifolin aglycone derived from domestic native Rhododendron mucronulatum, and an anti-obesity composition based on the same. The anti-obesity composition according to the present invention includes both a pharmaceutical composition for anti-obesity purposes and a food composition for prevention or improvement. In addition, the taxifolin glycoside and the taxifolin glycoside derived from Rhododendron mucronulatum japonica according to the present invention have effects of preventing and treating muscle loss, having an inhibitory effect on muscle loss, and can be applied to both a pharmaceutical composition and a food composition for prevention or improvement.

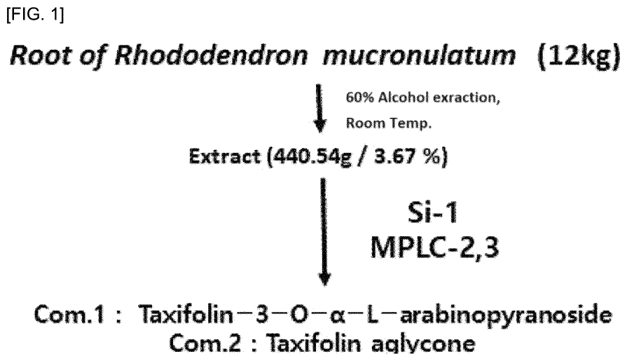

FIG. 1 is a view for explaining an extraction process according to an embodiment of the present invention.

Referring to FIG. 1, in one embodiment of the present invention, roots (12 kg) of Rhododendron mucronulatum were extracted with 60% alcohol at room temperature for 7 days, filtered through filter paper, and then the extract was concentrated under reduced pressure to recover (440.54 g). Then, the extract was dissolved in distilled water, filtered at filter paper., and then Silicagel column (40 μm, YAMAZEN, Osaka, Japan) was used, and the solvent was prepared in a ratio of Chloroform: Methanol: Water at 70:30:4 and proceeded to isocratic system, and spot was confirmed by TLC. In order to improve the purity of the target compound, MPLC (YAMAZEN, Osaka, Japan) separation purification was performed. ODS column (50 μm, YAMAZEN, Osaka, Japan) was used as the solvent, and water and Methanol were repeatedly performed with gradient system (0%→50% MeOH/20→80% MeOH). Finally, two kinds of compounds, that is, taxifolin-3-O-arabinopyranoside (Taxifolin-3-0-arabinopyranoside), which is a glycoside, and taxifolin, which is an aglycone, were finally separated and purified from an extract of Rhododendron mucronulatum japonica root, which will be described in more detail below.

### EMBODIMENT

### Supercritical Extraction

For the supercritical extraction of Rhododendron mucronulatum roots, research equipment for supercritical fluid extraction (ISA-SEFE-0500-0700-080, IIsinAutoclave, Daejeon, Korea) was used. Specifically, foreign substances in the sample were removed, washed, and dried to be used as experimental materials.

Each 100 g unit of the dried sample was pulverized to pass through a pulverization net of 200 mesh, and the temperature of the Rhododendron mucronulatum root sample was adjusted to 40-60° C. to maintain the temperature. Thereafter, when the temperature was stabilized, a Rhododendron mucronulatum root sample was added, CO2 gas was maintained at an equal pressure, and then the control valve was adjusted and injected until the temperature reached the experimental pressure condition of 400 to 600 bar through a high pressure pump at line.

After reaching the predetermined pressure, the extraction was performed by introducing alcohol of total edible alcohol into the lower part of the extraction bath at 5 mL or 10 mL per minute for 60 minutes or 240 minutes, and the extract (RM) was prepared by completing extraction by flowing CO2 using a high pressure pump at a pressure and temperature set to remove the remaining ethanol remaining in the sample for 30 min.

### Solvent Fractionation

After the supercritical extraction according to the above-described method, the supercritical extract residue of the Rhdondendron roots was recovered, and then the recovered extract was extracted into edible alcohol (30 to 100%) at room temperature for 3 days, followed by concentration under reduced pressure through filter paper filtration and freeze-drying to obtain a final Rhdondendron roots supercritical extract residue ethanol extract (RMSCFR). The obtained RMSCFR was dissolved in distilled water (primary or tertiary distilled water), filter paper filtration was performed, and an Ethyl acetate (EtOAC) layer and a water layer were secured using a separatory funnel, and at this time, the obtained EtOAC extract was a Rhdondendron sp. derived high content extract (RMRF).

### EXPERIMENTAL EXAMPLE 1

### TLC Analysis

In the present experimental example, TLC analysis was performed on the high content extract (RMRF) derived from Rhododendron mucronulatum paralea prepared by the above method.

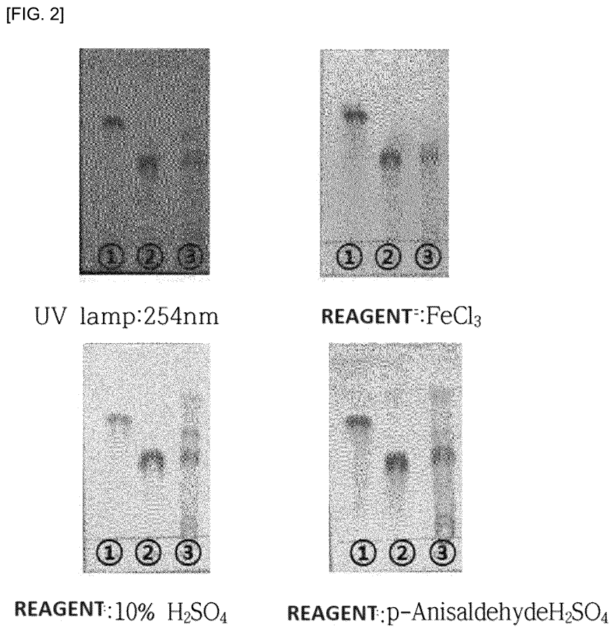

FIG. 2 is a TLC analysis result on a high content extract (RMRF) derived from Rhododendron mucronulatum.

Referring to FIG. 2, it may be confirmed that the taxifolin aglycone and the taxifolin glycoside are present in the RMRF fraction. That is, the chromatography results on (1) taxifolin aglycone, (2) taxifolin-3-O-arabinopyranoside, (3) RMRF I, which is a glycoside, show that (1) taxifolin aglycone, (2) taxifolin-3-O-Taxifolin-3-0-arabinopyranoside, which is a glycoside, are all present in the high content extract (RMRF) derived from Rhododendron mucronulatum according to the present invention.

### Total Phenol Content Analysis

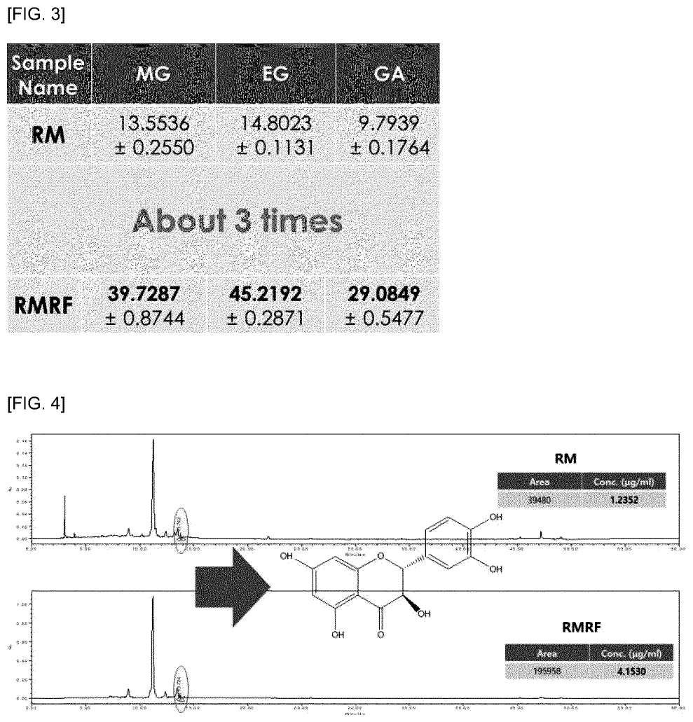

FIG. 3 is a total phenol content analysis result on a high content extract (RMRF) derived from Rhododendron mucronulatum.

Referring to FIG. 3, as a result of analyzing the content of total phenol (Methyl gallate, Ethyl gallate, Gallic acid), it can be seen that the total phenol content of the solvent-fractionated RMRF after extraction is three times higher than that of the extract RM. From these results, it can be seen that RMRF containing both taxifolin and taxifolin-3-O-arabinopyranoside can be expected to have strong physiological activity by the supercritical extraction of the Rhododendron mucronulatum roots according to the present invention.

### HPLC Analysis Result

For HPLC analysis, Waters 2695 Separation module, 2487 Dual λ Absorbance Detector was used, and SkyPak C18 analytical column (5 μM), Phenomenex KJ0-4282 guard column was used as the column, and 1% Formic acid (A), ACN (B) was used as the mobile phase (Gradient program: 10% B 0 min, 60% B 0-40 min, 100% B 40-45 min, 10% B 45-50 min, 10% B 50-60 min).

FIG. 4 shows the results of RMRF analysis of taxifolin aglycone with respect to RMRF and an alcohol extract (RM).

Referring to FIG. 4, taxifolin nonsaccharides were identified as 1.2352 ug/ml in a 60% Rhododendron mucronulatum root ethanol extract (RM) and 4.1530 ug/ml in a RMRF extract. Therefore, it can be seen that the content of taxifolin aglycone is increased by 336.22% or more compared to the conventional 60% ethanol extract.

FIG. 5 shows the results of RMRF analysis of taxifolin glycosides of RMRF and an alcohol extract (RM).

Referring to FIG. 5, it can be seen that the amount of taxifolin glycoside was increased by 684.17% compared to 60% of the ethanol extract as a result of analyzing the amount of taxifolin glycoside from the high content of taxifolin glycoside extract (RMRF) derived from Rhododendron mucronulatum.

### Structural Identification

### Taxifolin Aglycone

The final single compound was purified by repeated purification such as MPLC column chromatography from RM and RMRF, and NMR. and LC/MS data were measured in order to perform structural identification on the isolated and purified compound. The results are as follows.

White yellow amorphous powder Negative LC-MS: m/z 303.0 [M−H]−1H-NMR (300 MHz, DMSO-d6): δ 11.92 (1H, s, 5-OH), 6.75˜6.88 (3H in total, m, H-2′, H-5′ and H-6′), 5.92 (1H, d, J=2.1 Hz, H-8), 5.87 (1H, d, J=2.1 Hz, H-6), 5.00 (1H, d, J=11.1 Hz, H-2), 4.52 (1H, d, J=11.1 Hz, H-3).

13C-NMR (75 MHZ, DMSO-d6): 197.2 (C-4), 167.1 (C-7), 163.5 (C-5), 162.8 (C-9), 146.0 (C-4′), 145.1 (C-3′), 128.2 (C-1′), 119.6 (C-6′), 115.5 (C-5′), 115.2 (C-2′), 100.6 (C-10), 96.1 (C-6), 95.1 (C-8), 83.1 (C-2), 71.6 (C-3)

The structure of the taxifolin aglycone obtained therefrom is represented by the following Chemical Formula 1.

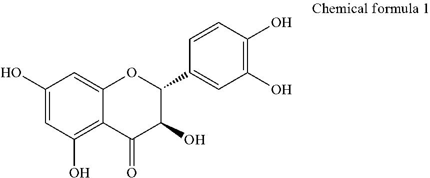 [SMILES: O=C1c2c(O)cc(O)cc2O[C@H](c2ccc(O)c(O)c2)[C@H]1O]

### Taxifolin Glycoside

The final single compound was purified from the Korean native Rhododendron mucronulatum extract by repeated purification such as MPLC column chromatography, and NMR and LC/MS data were measured to perform structural identification on the isolated and purified compounds.

White yellow amorphous powder, LC-MS, (positive-ion mode) m/z 437.1109 [M+H]+; ¹H-NMR, (700 MHZ, MeOH-d4) δ: 3.38 (1H, dd, J=11.2, 3.5 Hz, H-5″), 3.55 (1H, m, H-3″), 3.58 (1H, m, H-2″), 3.80 (1H, m, H-4″), 3.82 (1H, d, J=3.5 Hz, H-1″), 3.91 (1H, dd, J=11.2, 7.0 Hz, H-5″), 4.79 (1H, d, J=10.5 Hz, H-3), 5.12 (1H, d, J=10.5 Hz, H-2), 5.90 (1H, d, J=2.1 Hz, H-8), 5.92 (1H, d, J=2.1 Hz, H-6), 6.79 (1H, d, J=8.4 Hz, H-5′), 6.84 (1H, dd, J=8.4, 2.1 Hz, H-6′), 6.965 (1H, d, J=2.1 Hz, H-2′); ¹³C-NMR, (175 MHz, MeOH-d4) δ: 196.17 (C-4), 169.04 (C-7), 165.74 (C-5), 164.28 (C-9), 147.18 (C-4′), 146.58 (C-3′), 128.98 (C-1′), 120.79 (C-6′), 116.30 (C-5′), 116.00 (C-2′), 102.40 (C-1″), 101.40 (C-10), 97.41 (C-6), 96.42 (C-8), 83.83 (C-2), 76.67 (C-3), 73.23 (C-2″), 71.12 (C-3″), 66.79 (C-4″), 63.36 (C-5″).

The molecular weight confirmed through the final LC-MS can be confirmed to be taxifolin-3-O-α-L-arabinopyranoside of the following Chemical Formula 2, which is a glycoside form of the final taxifolin, as compared with the reference.

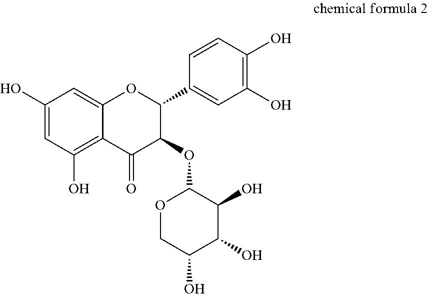

### EXPERIMENTAL EXAMPLE 2

The taxifolin glycoside or aglycone derived from Rhododendron mucronulatum japonica according to the present invention has an anti-obesity effect of inhibiting adipocyte differentiation. In the present experimental example, the efficacy of a natural extract (RM, RMRF) derived from native Rhododendron mucronulatum in Korea on the inhibitory activity against adipocyte differentiation was evaluated. To this end, 60% ethanol extract (RM) of Korean native plants, and taxifolin glycosides and aglycones, which are indicator and effective substances from RM, were used as test materials.

### Cell Culture

3T3-L1 cells, a mouse-derived preadipocyte, were purchased from the Korean Cell Line Bank and used. 3T3-L1 cells were cultured in a 37° C.-wet CO2 incubator (5% CO2/95% air) using a cell culture medium (complete DMEM culture medium) in which 10% bovine calf serum (BCF), 100 units/mL penicillin, and 100 μg/mL streptomycin were added to a DMEM medium (Welgene). When the cells were 80% full of the culture dish, the cell monolayer was washed with phosphate buffer saline (PBS, pH 7.4), and then the cells were detached by adding trypsin-2.65 mM EDTA and subcultured, and the medium was exchanged every 2 days.

### Measurement of Cell Viability

3T3-L1 cells were seeded into 24-well plate to 3×104 cells/well and the cells were cultured for 24 hours. After culturing the cells for 24 hours, the cells were cultured for 72 hours by exchanging with a cell culture solution containing a test substance. The cells were cultured for 72 hours, and then the number of living cells was measured by performing MTT assay (Denizot F and Lang R. J Immunological Method 89:271-277, 1986). The MTT assay method is based on the principle that dehydrogenase of mitochondria reduces MTT (Amresco) to produce blue formazan, and in this test, formazan was dissolved in isopropanol, and absorbance was measured at a wavelength of 570 nm.

### Differentiation Induction and Test Substance Treatment

3T3-L1 cells were seeded onto 24-well plate at a concentration of 1×105 cells/well. After the cells reached the confluence state, the cell culture medium was sequentially exchanged with three kinds of differentiation-inducing cultures (DM), and the cells were cultured to induce differentiation into adipocytes. That is, the cell culture medium was exchanged with a differentiation-inducing culture medium in which DMI (1 μM dexamethasone, 0.5 mM 3-isobutyl-1-methylxanthine (IBMX), 5 μg/mL insulin) was added to a DMEM medium containing 10% FBS to stimulate differentiation for 2 days. After 2 days, the cells were exchanged for a new differentiation-inducing culture medium in which 5 μg/mL insulin was added to DMEM medium containing 10% FBS to stimulate differentiation for another 2 days. After stimulating differentiation for a total of 4 days, cells were maintained in DMEM medium containing 10% FBS for 2 days to induce differentiation into adipocytes.

In order to investigate the effect of the test substance on adipocyte differentiation, the test substance was added to the differentiation-inducing culture medium and treated on the cells.

### Adipocyte Differentiation (Fat Accumulation) Measurement (Oil Red-O Staining)

After treatment with the test substance while inducing differentiation of 3T3-L1 cells, the cells were rinsed with DPBS (Welgene), and then the cells were fixed at room temperature for 1 hour by adding 4% paraformaldehyde (PFA, Biosesang). After the cells were fixed, the cells were treated with oil red O (Sigma-Aldrich) solution and stained at room temperature for 1 to 2 hours. After observing the staining degree of adipocytes visually, the cells were rinsed with distilled water and observed for adipocytes through a microscope.

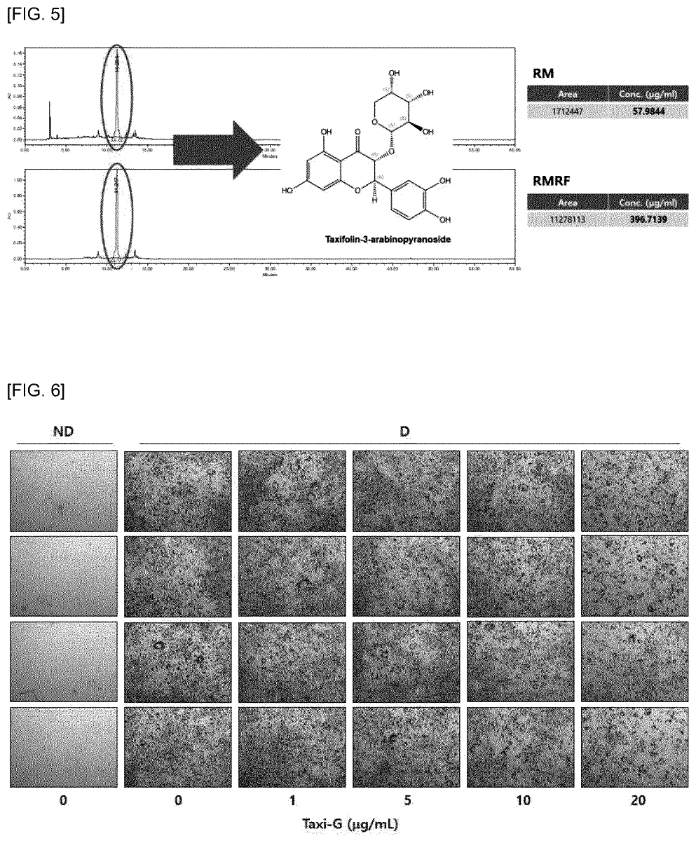

FIG. 6 is a photograph of cells observed when the “Taxifolin-3-O-arabinopyranoside” compound identified as a marker material and an effective material of the Rhdondendron sp. plants, is treated.

Referring to FIG. 6, it may be possible to significantly confirm the ability to inhibit adipocyte differentiation which increases in a concentration-dependent manner depending on the treatment concentration. In particular, at the concentration of 20 ug/ml, the highest concentration in the experiment, it was confirmed that the effect of inhibiting adipogenesis was suppressed by 40% or more.

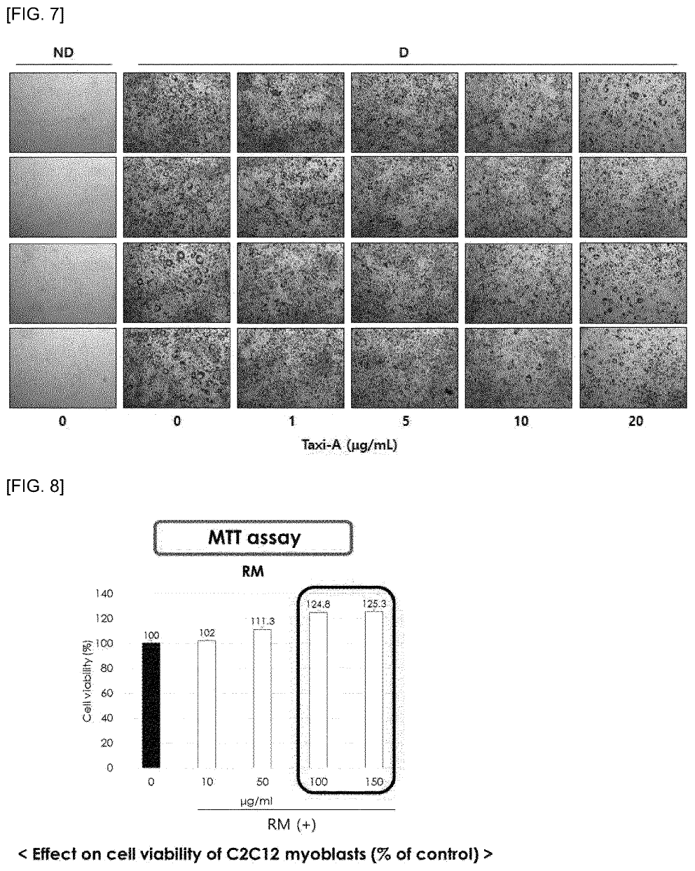

FIG. 7 is a photograph of a cell observed when a compound in the form of non-saccharide produced through enzymatic hydrolysis, that is, a compound “Taxifolin-aglycone” is treated from a glycoside compound well known as a marker material and an effective material derived from Rhododendron sp. plants, that is, “Taxifolin-3-O-arabinopyranoside”.

Referring to FIG. 7, it may be confirmed that the adipocyte differentiation inhibitory activity, which increases in a concentration-dependent manner depending on the treatment concentration, is significantly. In particular, at the concentration of 20 ug/ml, the highest concentration in the experiment, it was confirmed that the effect of inhibiting adipogenesis was suppressed by 40% or more.

The above results suggest that the Rhododendron sp. derived Taxifolin glycoside or non-saccharide (RM, RMRF) according to the present invention may be used as an anti-obesity therapeutic agent or an idle component of functional foods.

### EXPERIMENTAL EXAMPLE 3

The taxifolin glycoside or aglycone derived from Rhododendron mucronulatum japonica according to the present invention has a function of controlling muscle loss, which inhibits muscle loss as well as fat load. In this Experimental Example, the effect of the RMRF (RM) derived from native Rhododendron mucronulatum in Korea on muscle loss reduction prevention was evaluated. In particular, the protective effects of H2O2 and dexamethasone induced muscle cell damage in the in vitro system were tested. To this end, a 60% alcohol extract (RM) of Korean native plants, and a high-content extract (RMRF) with increased contents of taxifolin glycosides and aglycones, which are indicator and single compounds, from RM were used as test materials.

### Cell Culture

C2C12 cells, a myoblast derived from mouse skeletal muscle, were purchased from American Type Culture Collection (ATCC) and used. C2C12 cells were cultured in a 37° C.-wet CO2 incubator (5% CO2/95% air) using a cell culture medium in which 10% fetal bovine serum (FBS), 100 units/mL penicillin, and 100 μg/mL streptomycin were added to Dulbecco's Modified Eagle Medium (DMEM). When the cells were 80% full of the culture dish, the cell monolayer was washed with phosphate buffer saline (PBS, pH 7.4), and then the cells were detached by adding trypsin-2.65 mM EDTA and subcultured, and the medium was exchanged every 2 days.

### Measurement of Cell Viability

Cell viability of C2C12 cells was measured by MTT assay method (Denizot F and Lang R. J Immunological Method 89:271-277, 1986). C2C12 cells were seeded in 24-well plate at 2.5×104 cells/well and cultured for 24 hours. After culturing the cells for 24 hours, the cells were incubated with a cell culture medium treated with the test substance at various concentrations (0, 10, 50, 100, 150, 200 μg/mL) for 24 hours. The test materials were treated and incubated for 24 hours, and then the cell culture medium was exchanged with a 1 mg/mL MTT (Amresco) solution, the cells were further incubated for 2 hours, and then formazan formed in living cells was eluted with isopropanol to measure absorbance at 570 nm.

### Measurement of a Protective Effect on H2O2-Induced Muscle Cell Damage

C2C12 cells were seeded in 24-well plate at 2.5×104 cells/well and cultured for 24 hours. After culturing the C2C12 cells for 24 hours, the cells were treated with 100 μM H2O2 in order to induce muscle cell damage, and the cells were cultured for 24 hours by treating test materials with various concentrations together with 100 μM H2O2 in order to investigate the effect of protecting the muscle cell damage of each test material. The cells were cultured for 24 hours, and then MTT assay was performed in the same manner as described above to measure a cell viability.

### Measurement of a Protective Effect on Dexamethasone-Induced Muscle Cell Damage

C2C12 cells were seeded in 24-well plate at 2.5×104 cells/well and cultured for 24 hours. After culturing the C2C12 cells for 24 hours, the cells were treated with 500 μM dexamethasone in order to induce muscle cell damage, and the cells were cultured for 24 hours by treating the test materials with various concentrations together with 500 μM dexamethasone in order to investigate the effect of protecting the muscle cell damage of each test material. The cells were cultured for 24 hours, and then MTT assay was performed in the same manner as described above to measure a cell viability.

### Statistical Processing

All assay values were presented in mean+SEM. The collected results were analyzed using GraphPad Prism 5.0 (GraphPad software, San Diego, CA, USA) program. Student's t-test and one-way analysis variance (ANOVA) were used to compare the difference between the control group and the test substance treatment group. It was determined to be statistically significant only when p<0.05 or higher.

### Result

### Effect of a RMRF (RM) on Cell Viability

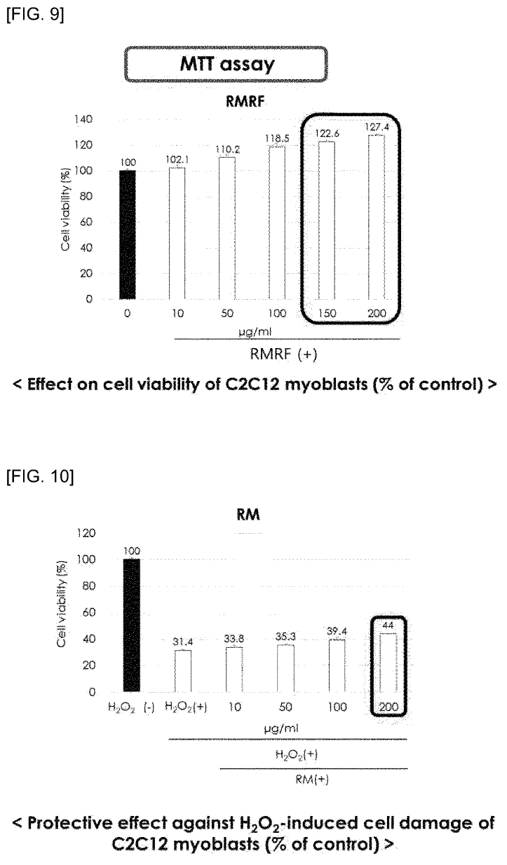

FIGS. 8 and 9 are the results of analyzing the cell viability of taxifolin glycosides (RM) and taxifolin aglycones (RMRF), respectively.

Referring to FIGS. 8 and 9, in order to examine cytotoxicity of two kinds of natural extracts (RM, RMRF) in C2C12 cells, natural extracts of various concentrations (0, 10, 50, 100, 150, 200 μg/mL) were treated in a cell culture medium, cultured for 24 hours, and then subjected to MTT assay. Cell viability of C2C12 cells was increased by treatment with RM and RMRF at various concentrations (10, 50, 100, 150, 200 μg/mL). When the concentration of RM was 50 to 200 μg/mL, the cell viability was increased by 11.3%, 24.8%, and 25.3%, respectively, compared to the control group (0 μg/mL).

Similarly, when the treatment concentration of RMRF was increased, the cell viability was increased, and when the treatment was performed at a concentration of 50 to 200 ug/mL, the cell viability was increased by 10.2%, 18.5%, 22.6%, and 27.4%, respectively, as compared to the control group (0 μg/mL). According to the above results, the domestic native Rhododendron mucronulatum extracts including taxifolin glycosides and taxifolin aglycones are not toxic to normal muscle cells, and muscle reduction can be expected by increasing muscle cells.

### H2O2 (Hydrogen Peroxide) Analysis Result

FIG. 10 and FIG. 1 show the results of analyzing cell viability when taxifolin glycoside (RM) and taxifolin aglycone (RMRF) for H2O2 (hydrogen peroxide) were used, respectively.

H2O2 (hydrogen peroxide) induces oxidative stress (oxidative stress) in the in vitro system with a strong oxidizing agent, and in order to investigate the effect of RM, RMRF on the muscle cell damage caused by oxidative stress, the cell culture solution of C2C12 cells was treated with 100 μM H2O2 to induce oxidative stress, and cultured with RM, RMRF, and then the cell viability of C2C12 cells was measured.

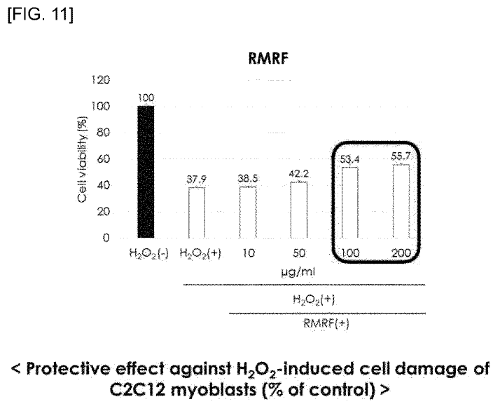

Referring to FIGS. 10 and 11, when treated with H2O2, cell viability was significantly reduced compared to the control [H2O2 (−)/(−)] not treated with H2O2.

When RM was treated at various concentrations (10, 50, 100, and 200 μg/mL), cell viability was significantly increased compared to the control [H2O2 (+)/(−)] treated only with H2O2. When RM was treated at a concentration of 200 μg/mL, cell viability was increased by 44% compared to the control group treated with only H2O2 [H2O2 (+)/(−)]. When RMRF was treated at concentrations of 10, 50 and 100, 200 μg/mL, cell viability was significantly increased compared to the control group treated only with H2O2 [H2O2 (+)/(−)]. Particularly, in the concentration-treated group of 100 to 200 μg/mL, it was confirmed that the cell viability was increased by 53.4% and 55.7%, respectively, as compared to the control group [H2O2 (+)/(−)] treated only with H H2O2. From the above results, it could be seen that the domestic native Rhododendron mucronulatum extracts including taxifolin glycosides and taxifolin aglycones inhibited muscle loss caused by apoptosis of muscle cells induced by oxidative stress.

### Dexamethasone Analysis Result

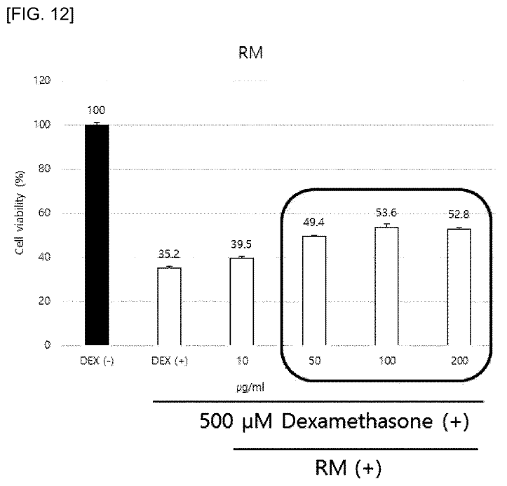

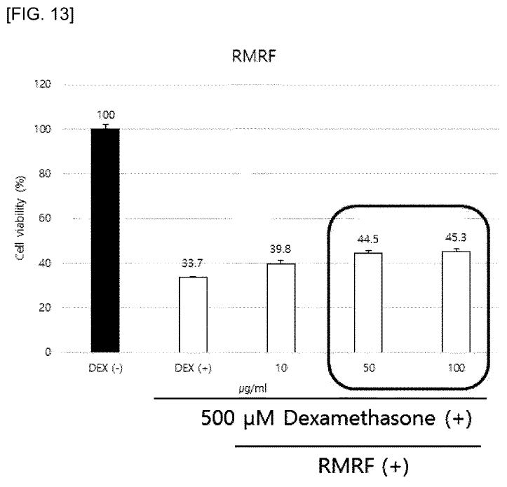

FIG. 12 and FIG. 13 show the results of analyzing the cell viability when taxifolin glycoside (RM) and taxifolin aglycone (RMRF) were used for dexamethasone, respectively.

Dexamethasone is one of the representative glucocorticoid, which causes the degradation of skeletal muscle in clinical misuse, and based on this, it is widely used to induce muscle cell damage in the in vitro system. In order to investigate the effect of the RM and RMRF according to the present invention on the muscle cell damage caused by glucocorticoid, the cell culture fluid of C2C12 cells was treated with 500 μM dexamethasone to induce the muscle cell damage, and the cell viability of C2C12 cells was measured after being cultured by the treatment of RM and RMRF.

Referring to FIGS. 12 and 13, when treated with Dexamethasone, cell viability was significantly reduced compared to the control [DEX (−)/(−)] not treated with dexamethasone.

It was confirmed that RM at a concentration of 10 to 200 μg/mL significantly increased the decrease of cell viability induced by dexamethasone. That is, as the concentration of 10 to 200 μg/mL was increased, the protective effect on the cell damage was statistically significantly increased, and it was confirmed as 39.5%, 49.4%, 53.6%, and 52.8%.

In addition, when RMRF was treated at the concentrations of 10, 50, and 100 μg/mL, the cell viability was significantly increased compared to the control [DEX (+)/(−)] treated only with dexamethasone. It was confirmed that RMRF at various concentrations (10, 50, 100 μg/mL) had a significant effect on cell viability decrease induced by dexamethasone. That is, as the concentration was increased, cell viability was increased to 39.8%, 44.5%, and 45.3%. From the above results, it can be seen that the domestic native Rhododendron mucronulatum extract including taxifolin glycosides and taxifolin aglycones inhibits muscle loss caused by the breakdown of skeletal muscle and the apoptosis of muscle fiber cells, which are one of the side effects of glucocorticoid-based drugs.

### INDUSTRIAL APPLICABILITY

It is a composition for preventing and treating obesity and muscle loss, and has industrial applicability.

1. A pharmaceutical composition for preventing and treating obesity and muscle loss, the pharmaceutical composition comprising rhododendron mucronulatum extract as an active ingredient.

2. The pharmaceutical composition for preventing and treating obesity and muscle loss of claim 1, wherein the Rhododendron mucronulatum extract is a feature comprising a taxifolin glycoside or a taxifolin aglycone.

3. The pharmaceutical composition for preventing and treating obesity and muscle loss of claim 2, wherein the Rhododendron mucronulatum extract is obtained by supercritical extraction of Rhododendron root, and is a feature.

4. The pharmaceutical composition for preventing and treating obesity and muscle loss of claim 2, wherein the taxifolin glycoside comprises a compound represented by Formula 1 below as a feature,

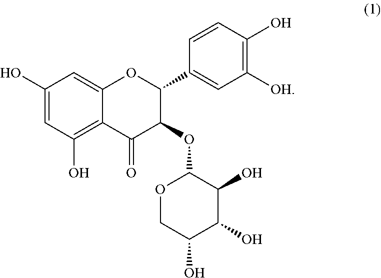

5. The pharmaceutical composition for preventing and treating obesity and muscle loss of claim 2, wherein the taxifolin glycoside comprises a compound represented by Formula 2 below as a feature,

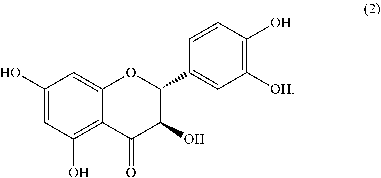 [SMILES: O=C1c2c(O)cc(O)cc2O[C@H](c2ccc(O)c(O)c2)[C@H]1O]

6. A food composition for preventing obesity and muscle loss, comprising Rhododendron mucronulatum extract as an active ingredient.

7. The food composition for preventing obesity and muscle loss of claim 6, wherein the Rhododendron mucronulatum extract comprises a taxifolin glycoside or a taxifolin aglycone.

8. The food composition for preventing obesity and muscle loss of claim 7, wherein the Rhododendron mucronulatum extract is obtained by supercritical extraction of Rhododendron mucronulatum roots.

9. The food composition for preventing obesity and muscle loss according to claim 7, wherein the taxifolin glycoside comprises a compound of the following formula (1),

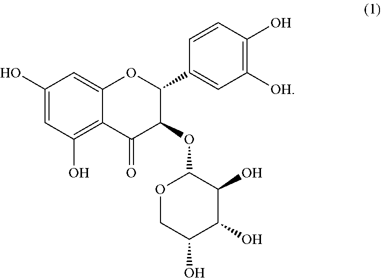

10. The food composition for preventing obesity and muscle loss according to claim 7 wherein the taxifolin glycoside comprises a compound of the following formula (2),

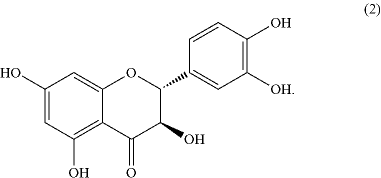 [SMILES: O=C1c2c(O)cc(O)cc2O[C@H](c2ccc(O)c(O)c2)[C@H]1O]

11. A food composition for preventing obesity and muscle loss, the food composition comprising at least one selected from the group consisting of compounds of Formulae (1) and (2) below.

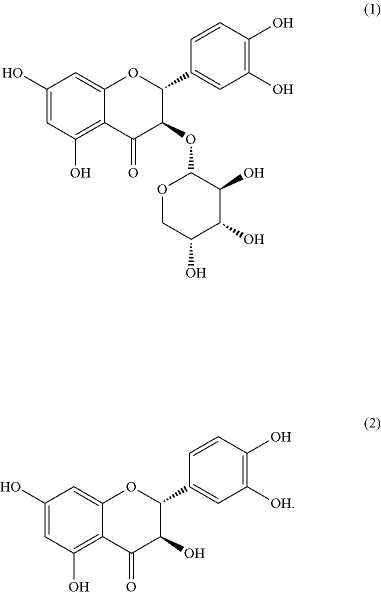

12. The food composition for preventing obesity and muscle loss of claim 11, wherein the compound is a feature which is extracted from the roots of Rhododendron mucronulatum.

13. The food composition for preventing obesity and muscle loss of claim 11, wherein the food composition for preventing obesity and muscle loss comprises an animal feed additive as a feature.

14. (canceled)

15. (canceled)
